(19) United States
(12) Patent Application Publication
(10) Pub. No.: US 2003/0211487 A1
(43) Pub. Date: Nov. 13, 2003

(54) High efficiency mapping of molecular variations to functional properties

(76) Inventor: Herschel Rabitz, Lawrenceville, NY (US)

Correspondence Address: Peter J Butch III, Synnestvedt & Lechner, 2600 Aramark Tower, 1101 Market Street, Philadelphia, PA, 19107-2950, US

(21) Appl. No.: 10/169,479

(22) PCT Filed: Jan. 3, 2001
(86) PCT No.: PCT/US01/00150

(51) Int. Cl. C12Q 1/68; G01N 33/53; G06G 7/48; G06G 7/58; G06F 19/00; G01N 33/48; G01N 33/50

(52) U.S. Cl. 435/6; 435/7.1; 703/11; 702/19

(57) ABSTRACT

A method for the selective variation of a multi-variable molecular synthesis to optimize a reaction product functional property, including the steps of: constructing a first order library of functional property output data obtained by reacting all first order library input variable combinations for the multi-variable synthesis and measuring the functional property for every reaction product, wherein the first order library input variable combinations include all values selected for each variable taken one at a time; ordering the values for each input variable according to their effect upon functional property optimization; constructing a second order library of functional property output data obtained by reacting a set of second order library input variable combinations coarsely sampled two variables at a time from the ordered input variables and measuring the functional property for every reaction product; and interpolating among the functional property output data for functional property optimization.

### CROSS-REFERENCE TO RELATED APPLICATION

The present application claims priority benefit under 35 U.S.C. § 119(e) of U.S. Provisional Patent Application Serial No. 60/174,225 filed Jan. 3, 2000. The disclosure of this application is incorporated herein by reference.

### BACKGROUND OF THE INVENTION

In the chemical sciences, many laboratory experiments, environmental and industrial processes, as well as modeling exercises, are characterized by a large number of input variables. A general objective in such cases is an exploration of the high-dimensional input variable space as thoroughly as possible for its impact on observable system behavior, often with either optimization in mind or simply for achieving a better understanding of the phenomena involved. An important concern when undertaking these explorations is the number of experiments or modeling excursions necessary to effectively learn the system input/output behavior.

In the performance of experiments or the modeling of chemical/physical systems where there are large numbers of input variables accessible for alteration, the alteration of the input variables may be done with some design strategy in mind, or it may occur randomly because of natural uncontrolled variations in the input In either circumstance, a common goal is to perform as many runs as possible, aiming at an exploration of the input variable space with respect to its impact on one or more system observables of interest. Such exercises may be performed either to gain a physical understanding of the role of the input variables, or often, ultimately for purposes of optimization to achieve one or more desired physical objectives by special choice of the input variables.

Molecular materials (i.e., a sample consisting of a single type of molecule) encompass many applications, including mutated proteins and pharmaceuticals. For a molecular material, the i-th variable may be the i-th site for chemical functionalization on a reference molecular structure. In the case of a protein subject to amino acid mutation, the total number of variables (i.e., sites for mutation) can be very large, and variable xᵢ associated with backbone site i may take on up to 20 values over the naturally occurring amino acids. In contrast, pharmaceutical molecules are typically of modest size, generated by functionalization of a small number of sites on a reference chemical scaffold. For pharmaceuticals, the i-th site variable could take on a large number of values, as a rather arbitrary set of chemical moieties may be considered for substitution on a suitable molecular scaffold.

Molecular materials inherently differ from those of mixture formulations, as molecular moiety input variables are discrete (e.g., methyl, ethyl, chloro, etc.) While the component mole fractions as input variables in mixtures can take on continuous values. Mixture materials drawn from a large set of possible molecular species have both discrete and continuous variables. All of these material problems, characterized by either large numbers of input variables or large numbers of variable values, has led to much interest in high throughput synthesis and screening techniques in an attempt to deal with the potentially exploding number of material samples that maybe generated.

A common characteristic of all the aforementioned illustrations, and many others, is the large number of variables that may naturally arise to describe the input. The notion of “large” in this context depends on the particular application, and especially the difficulty of either appropriately observing or calculating the system output corresponding to any single specification of all of the input variables. The search for pharmaceuticals is generally of a similar nature involving low numbers of variables (i.e., the sites for functionalization on a molecular scaffold), but the number of moiety values for each of these variables can be very large, ranging up to 10² or more. In this case, making one potential pharmaceutical molecule may be easy, but making all relevant possibilities gets out of hand. In other problems, the number of variables involved can be inherently large, and one example occurs where the input is a function and good resolution is required, thereby leading to hundreds or more of discretized input variables.

Thus, the discovery of molecular materials with desired properties continues to present a heavy burden because of the exponential relationship between the number of molecular variations within a system and the number of possible variable combinations within a system that must be explored. Techniques of combinatorial chemistry have attempted to address this problem through random or quasi-random synthesis approaches. Alternatively, modeling procedures have attempted to achieve guidance by design. None of these techniques has come up to its full promise and capabilities. There remains a need for methods for organizing and interpreting data so that the exploration of multi-variable reaction systems may be made more feasible.

### SUMMARY OF THE INVENTION

This need is met by the present invention. It has now been discovered that when a modest initial number of experiments is used to specifically guide further experimentation by the techniques employed by the present invention, multi-variant reaction systems may be explored in a very efficient fashion without reacting all possible variable combinations within the system. The present invention incorporates the discovery that by breaking down the impact of each variable into a hierarchy of cooperative terms, and recognizing that the contribution of any particular variable can be gauged directly from laboratory observations based on a rational ordering of the molecular variables, the number of necessary molecular synthesis experiments for the performance of a thorough investigation of a multi-variant reaction system grows, at most, polynomially with the number of sites and, under certain conditions, is actually invariant to the number of sites.

Therefore, according to one aspect of the present invention a method for the selective variation of a multi-variable molecular reaction to optimize a functional property of the reaction product is provided, which method includes the steps of:

constructing a first order library of functional property output data obtained by reacting a all first order library input variable combinations for the multi-variable synthesis and measuring the functional property for every reaction product, wherein the first order library input variable combinations include all values selected for each variable taken one at a time while the other variable values are held constant or randomized;

ordering the values for each input variable according to their effect upon functional property optimization based upon the first order library functional property output data;

constructing a second order library of functional property output data obtained by reacting a set of second order library input variable combinations coarsely sampled from the ordered input variables and measuring the functional property for every reaction product, wherein said second order library input variable combinations are assembled two variables at a time from said coarse sampling of ordered input variables while the other variable values, if any, are held constant or randomized; and

interpolating among the functional property output data for optimization of the functional property.

The second order library may be constructed either by reacting an ordered coarse sampling of the ordered variable values, a fully random coarse sampling of the ordered variable values, by a combination of the two sampling techniques, or by other coarse sampling techniques. Which sampling technique to employ will depend upon the number of variables and laboratory synthesis techniques.

The inventive method is a variation of and improvement upon conventional High Dimensional Model Representation (HDMR) algorithms. HDMR employs a critical set of organizing principles to directly guide laboratory synthesis studies resulting in the rapid identification of compounds with desired functional properties. The present invention improves upon HDMR techniques by using an ordered sampling of the synthesis reaction variables to guide the second stage of modest synthesis studies, ultimately leading to a means for quantitatively estimating the functional properties of molecules throughout the full space of possibilities, including those that have not been synthesized.

The inventive method thus first performs a initial set of syntheses involving all values selected for the reaction variables and the observation of their functional impacts, followed by algorithmic analysis of the results in which the variable values are ordered according to their effect upon the optimization of the functional property of interest for the reaction product, to then suggest the performance of further selective sets of ordered or randomly sampled molecules. The overall information from these syntheses and their functional impact is then reassembled into a High Dimensional Model Representation, to interpolate functional properties throughout the entire space of molecular possibilities. Cases with millions or more molecular possibilities can be quantitatively investigated with as few as a couple of hundred judiciously synthesized molecules, as an example of the potential savings.

Other features of the present invention will be pointed out in the following description and claims, which disclose the principles of the invention and the best modes that are presently contemplated for carrying them out.

### BRIEF DESCRIPTION OF THE DRAWINGS

A more complete appreciation of the invention and many more other intended advantages can be readily obtained by reference to the detailed description of the invention when considered in connection with the drawing figures, wherein:

FIG. 1, is a flow chart depicting a method according to the present invention;

FIG. 2A is a histogram plot of stability data from mutations of the gene V protein at sites I47 and V35, organized as originally presented;

FIG. 2B is a rearrangement of the data of FIG. 2A performed by a method according to the present invention;

FIG. 3A is the same as FIG. 2A, but for DNA binding affinity;

FIG. 3B is an analogous rearrangement of the data of FIG. 3A;

FIG. 4A is a histogram plot of phenotypic data from mutations of gene V protein at sites I47 and V35, organized as originally presented; and

FIG. 4B is an analogous rearrangement of the data of FIG. 4A.

### DETAILED DESCRIPTION OF THE PREFERRED EMBODIMENT

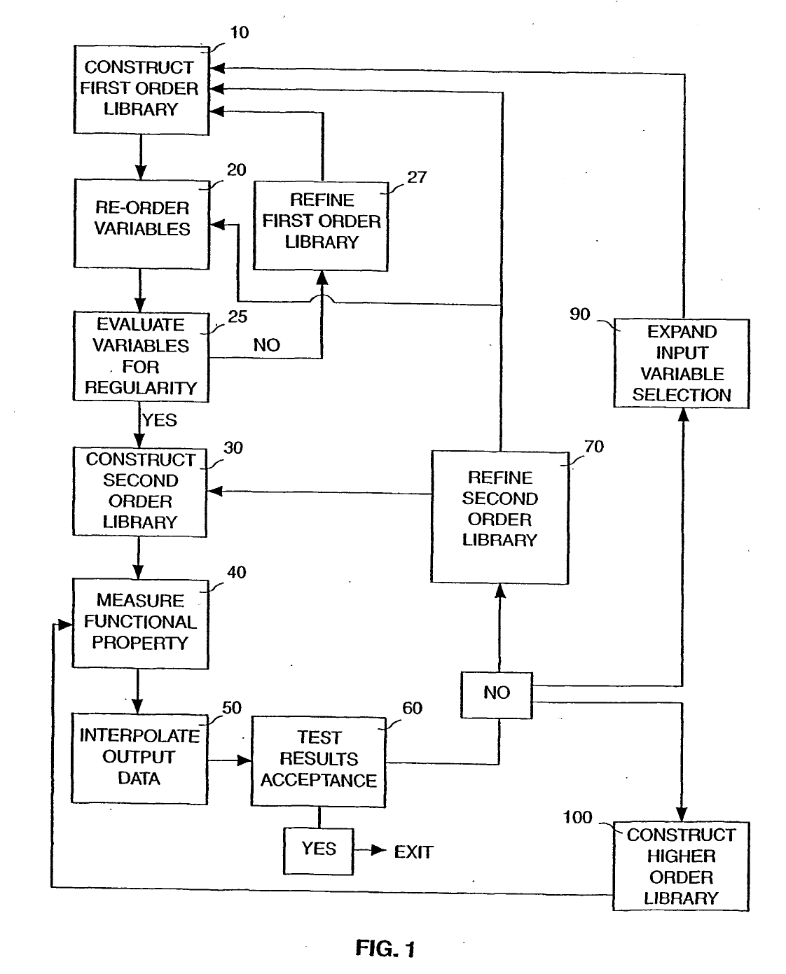

FIG. 1 depicts an applications program embodying the method of the present invention for the discovery of a molecular material, for example, a reference molecular structure being investigated for pharmaceutical activity. In step 10, a first order library of functional property output data is constructed by means essentially conventional to HDMR. For a multi-variable reaction containing variables a, b, c . . . z, values for each variable are selected a₁, a₂, a₃ . . . a_{N}; b₁, b₂, b₃ . . . b_{N}, and so forth. Input variable combinations are prepared taking each variable one at a time while the other variables of the reaction are held constant or randomized. Thus, combinations would be prepared for variables a₁, a₂, a₃ . . . a_{N}, while variables b through z are held constant or randomized, and so forth for all variables of the reaction. One of the preferred embodiments of the present invention, which represents an improvement to convention HDMR techniques, is to fully randomly sample the overall space to form the input variable combinations, with the only requirement being that all of the selected variable values be included among the sampled combinations. Regardless of how the input variable combinations are formed, the combinations represent a significant reduction in the total number of combinations that would have to be reacted in order to explore all possible combinations.

The first order library is constructed by reacting each combination and then measuring the functional property or properties of interest for every reaction product. The objective is to initially react all selected values for each variable, taken one at a time while the other variable values are held constant or randomized to obtain a one-dimensional sampling through a multi-dimensional space for each variable that provides a measurement of the effect its selected values have upon the functional property of interest.

For a molecules being investigated for pharmaceutical activity, the variable pool may include one or more chemical scaffolds, i.e., the basic molecular structure or frame-work, having one or more sites for chemical functionalization, as well as the various chemical moieties selected for functionalization of the sites (e.g., methyl, ethyl, chloro, etc.) The variables may also include structural variations and spatial features of the scaffold. Functional properties to be optimized include pharmaceutical activity, minimization of side-effects, bioavailability, improved product yield, and other properties essential to targeted drug discovery.

Thus, for purposes of the present invention, the “value” of a variable does not necessarily refer to an empirical quantity, but rather to the identity of the various options under consideration. If variable a represents the chemical scaffold on which moieties are to be varied at sites of chemical functionalization, then a₁, a₂, a₃ . . . would represent the scaffold structures to be investigated. If variable b represents the moiety to be varied at a first site of chemical functionalization on the scaffold, then b₁, b₂, b₃ . . . b_{N} would represent the various moieties selected for testing at this point of functionalization, such as chloro, fluoro, methyl, ethyl, and so forth. Other variables and values would be assigned to the remaining chemical functionalization sites.

The present invention is not limited to the investigation of molecules for pharmaceutical activity, but is applicable to molecular materials in general. Non-limiting examples of molecular applications of interest besides those having pharmaceutical utilization include molecules having optical activity, magnetic activity, electrical activity, viscoeleastic activity, and the like. As with the molecules being investigated for pharmaceutical activity, the variable pool will include one or more chemical scaffolds, i.e., the basic molecular structure or frame-work, having one or more sites for chemical functionalization, as well as the various chemical moieties selected for functionalization of the sites. The present invention is also applicable to polymeric materials in general, which consist of discrete monomer moieties, each consisting of a chemical scaffold having one or more sites of chemical functionalization capable of being varied for purposes of the present invention. The functional property to be evaluated would be a functional property of the polymer.

Another example of a multi-variable system for use with the method of the present invention is a candidate protein for site-directed mutagenesis. Each amino acid within the protein sequence represents a potential independent variable chosen from among the amino acids employed in protein synthesis. The functional property to be optimized would be a property of the protein such as folding stability, ligand binding affinity, and the like.

The variables may be derived from an initial combinatorial chemistry screening of potential molecular structures, or from pharmaceutical leads. For example, a naturally-occurring compound discovered to have therapeutic properties would provide the chemical scaffold to be optimized, as well as related scaffolds to be investigated, and lead to the selection of chemical moieties to be varied at the chemical functionalization sites of each scaffold, with the objective being to optimize the therapeutic effect in term of potency, efficacy, safety, bioavailability, and the like. Another example of a pharmaceutical lead would be a protein discovered to have a ligand binding affinity producing a therapeutic effect, which would provide an amino acid sequence for site-directed mutagenesis at one or more sequence positions, with the objective being to improve the ligand binding ability to enhance the therapeutic effect.

Molecular materials are examples of systems in which the input variables have discrete values. Other systems to which the present invention is applicable may have variables with continuous values, such as the component mole fractions of chemical mixtures. Reaction conditions such as temperature, pressure, reaction time, and the like may also introduce continuous value input variables to variable combinations that would otherwise be limited to variables having finite and discrete values. Continuous value variables require the selection of data points that provide a complete sampling of the entire variable continuum. Regardless of whether the variables within a multi-variable system are continuous or discrete, the objective is to initially react all selected values for each variable, taken one at a time while the other variable values are held constant or randomized.

In step 20, the first order library output data is used to order the values for each variable in such a way that the property under consideration varies monotonically with the variable values, so that the values are ordered based upon their effect upon the functional property. In other words, the functional property is permitted to dictate the ordering of the selected values, with the selected values for each variable being ranked from the perspective of the functional property as an observer, so that the value producing the most optimum functional property is assigned the greatest significance, and so forth down to the value producing the least optimum functional property. The objective is to identify the “natural” order of values for each variable, with “natural” being defined as the rational ordering of the variable values based upon actual experience with respect to the functional property.

It should be noted that steps 10 and 20 can be applied to more than one functional property, with each property having its own natural ordering of variables. One objective could be to find the reaction product with the optimum combination of two or more functional properties, in which case step 10 would include the measurement of the two or more properties and step 20 would essentially score each variable value based upon the results achieved with respect to the optimumization for each property. Each functional property could be weighted differently for variable value scoring purposes depending upon its importance to the ultimate objective sought for the optimized reaction product.

In step 25, the ordered variables are evaluated to confirm that the output data for each variable exhibits more or less regular behavior over the range of values sampled. For purposes of the present invention, “regular” is defined as the condition where the functional property differences between the nearest variable values are “smooth,” i.e., as small as possible. If not, the method proceeds to step 27, wherein the first order library is refined. Refinement can be accomplished in several ways, with the objective being to expand the set of first order library input variable combinations. One way to accomplish this would just be to repeat step 10 to obtain another set of output data by forming new input variable combinations, again taking each variable one at a time while the other variables are held constant or randomized. Another way to accomplish this is to expand the number of variable values. Using the example of the molecular structure being investigated for pharmaceutical activity, this would include the exploration of another chemical scaffold, or the evaluation of additional moieties at the sites of chemical functionalization. The method would then repeat steps 10 (reacting the combinations and measuring the functional property), 20 (ordering the variable values from the perspective of the functional property) and 25 (evaluating the ordered variable values for regularity or smoothness), followed by step 27 again, if necessary.

Another way to expand the set of first order library input variable combinations would be to obtain full or partial second order output data and introduce this to the reordering of the variable values. In other words, some or all of the first order library input variable combinations would be reassembled taking variable values two at a time while the other variable values are held constant to obtain a more thorough sample of the possible variable combinations and a better assessment of cooperation among variables. Steps 10, 20 and 25 would again be repeated, also followed by step 27 again, if necessary. With either option, the repetition of steps 10 and 20 may result in a re-ordering of the values of at least one of the variables as the increased data may provide new insight into the rational hierarchy of the variable values from the perspective of the functional property, which in turn may result in a “smoother” or more regular ordering of the data.

Once step 25 has confirmed that the ordered variable values are as regular as possible the method proceeds to step 30, wherein a second order library of functional property output data is obtained by reacting a set of second order library input variable combinations that are coarsely sampled from the ordered variable values of step 20. For purposes of the present invention, “coarse” sampling is defined as a modest partial sampling guided by the observation of the impact the value for each variable has upon functional property optimization that is effective to permit quantitative estimation of the functional properties for reaction products throughout the full space of possibilities, including those that have not been synthesized. Coarse sampling represents an economy of scale in comparison to having to sample all possible variable combinations to arrive at the reaction product with the optimum functional property.

The coarse sampling may be performed by one of several ways, with the objective being to obtain a reasonable estimate of functional property performance over the full space of possibilities. For example, an ordered sampling can be performed wherein the ordered variable values are assembled along multi-dimensional axes or in a multi-dimensional array and sampled periodically, so that every fifth, tenth, twentieth or hundredth variable combination is sampled. Increasing the number of combinations that are sampled consequently increases the resolution that is obtained over the variable space. Alternatively, a completely random sampling of the variable space can be performed. Ordered or random sampling may be performed either uniformly or non-uniformly over the full space of possibilities. The non-uniform sampling would be performed iteratively until regions of optimum functional property performance were identified.

Coarse sampling techniques for second order library construction are otherwise essentially conventional to HDMR mapping, are readily employed by those skilled in the art, and do not require detailed explanation. The primary contribution of the present invention resides not in the coarse sampling technique, but in the rational, natural ordering of the variable values from the perspective of the functional property, which makes possible the coarse sampling in the first place. It is clear that once this concept is understood by reference to the present specification, those of ordinary skill in the art shall be able to modify existing HDMR software algorithms without undue effort to accomplish the goals described herein.

Conventional coarse sampling techniques, including those known as cut-HDMR and RS-HDMR, are disclosed in Rabitz et al., “General Foundations of High Dimensional Model Representation,” J. Math. Chem., 25, 197-233 (1999). Cut-HDMR employs regular coarse sampling around a reference point, referred to as a cut center, while RS-HDMR determines the expansion functions by coarse Monte Carlo sampling over the entire multi-dimensional space.

The second order library construction is otherwise essentially conventional to HDMR. Being a second order library, the input variable combinations are assembled taking two variable values at a time with the choice of the other variables depending upon the coarse sampling technique. The variable combinations are then reacted to obtain reaction products, on which functional property measurements are performed.

Step 40 represents the functional property measurement step. Functional property measurements are performed for the reaction product of every second order library input variable combination, to obtain a complete set of output data for the second order library.

The method proceeds to step 50, wherein the output data from the first and second order libraries is interpolated to identify the combination of input variable values producing the reaction product with the optimum functional property. This again is a step that is essentially conventional to HDMR. In all likelihood the input variable combination that is identified was never reacted, in which case the method proceeds to step 60 for the reaction of the variable combination identified as producing the reaction product with the optimum functional property. Step 60 represents a testing of the results to determine if the outcome based upon interpolation of the functional property output data in its present form is acceptable. If the results are acceptable, the method is complete, because the reaction product with the optimum functional property of interest has been identified. If the results are not acceptable, several options remain.

The first is to proceed to step 70, for refinement of the second order library. This essentially involves more coarse sampling to increase the output data resolution for the second order library and obtain more data point for interpolation. For example, if an ordered sampling of every twentieth combination had been performed, one could sample every tenth combination to obtain more data points. If the input variable combinations had been randomly sampled, then additional random sampling can be performed to obtain more data points. Had the random sampling been non-uniform, then other regions in the space of possibilities can be non-uniformly randomly sampled until the region with optimum functional property performance is identified.

The second order library can also be refined by making correlated input variable combinations based on the information at hand. If each variable is represented as a coordinate axis in multi-dimensional space with the selected variable values positioned thereon according to their natural order as perceived by the functional property, then the correlated combinations essentially represent a rotation of the coordinate axes to regions that coincide with optimum functional property performance by matching as closely as possible the perceived natural variable values for two or more of the variables. In essence, this represents a multi-dimensional cut across the space of possibilities to collect additional data points by exploration of regions of possible optimum functional property performance.

The present invention contemplates the iterative repetition of steps 30, 40, 50, 60 and 70 until enough output data is obtained for the interpolation step to identify the reaction product with the optimum functional property. At any point in this process, however, whether steps 30 through 70 have been performed once or many times, it may be desirable to repeat step 20. That is, the second order library output data may reveal a more rational ordering of the natural values of the variables from the perspective of the functional property that could not be identified with the information at hand when the initial ordering was performed. The method may proceed directly back to repeat step 20 from steps 30 through 70, or it may first proceed back to repeat step 10 for construction of another set of first order library output data to obtain additional data points to use with the information at hand when repeating step 20.

Steps 30 through 70 can then be repeated with additional coarse sampling based on the variable value hierarchy, if any, produced by the repetition of step 20. The iterative repetition of steps 30 through 70 may be continued, including returns to step to for reassessment of the rational ordering of the natural variable values until the reaction product with the optimum functional property is identified.

One consequence may be that even though the reaction product with the optimum functional property is identified over the entire space of possibilities as defined by the values selected for each variable, the optimum value may still fall short of the target value set at the outset of the investigation. Using the example of the molecular structure being investigated for pharmaceutical activity, the compound identified as having, for example, the highest potency among all the possible combinations, may still have a potency too low to merit further study as a candidate drug. Most likely this is a consequence of the values selected for one or more variables, with the solution being to proceed to step 90 of the inventive method, wherein the variable value sets are expanded and the method repeated from the beginning.

That is, in step 90 provides the option of repeating the inventive method after expanding all or some of the variable value sets. For example, additional chemical scaffolds could be selected for investigation, or additional chemical moieties could be selected for one or more of the sites of chemical functionalization. The objective is to expand the space of possibilities in search of a region of functional property optimization within the goals targeted for the functional property.

Another consequence may be that it is simply not possible to accurately interpolate over the entire space of possibilities based on first and second order library output data alone because of third and possibly fourth order cooperativity. Chemical systems are ordinarily defined by low order multi-variable cooperativity, so that in most cases first and second order library output data is sufficient to obtain sufficient information to permit interpolation over the space of possibilities to identify the reaction product with the optimum functional property. However, on occasion, and particularly with systems containing a large number of variables, the opportunities for variable interdependence increases, requiring the exploration of third and possibly fourth order variable value combinations and the output data derived therefrom, and so on.

This is depicted in FIG. 1 as step 100, which identifies the construction of third order and higher libraries of functional property output data derived from the reaction of input variable combinations coarsely sampled from the output data of the prior library of output data. That is, the third order library would be based on variables taken three at a time coarsely sampled from the output data of the second order library, the fourth order library would be based on variables taken four at a time coarsely sampled from the output data of the third order library, and so forth. At any point the output data may be interpolated according to the method of the present invention for identification of the reaction product with the optimum functional property.

Certain aspects of the inventive method are illustrated by reference to the following depiction of its application to site-directed protein mutagenesis, which should not be interpreted as limiting the scope of the invention as it is defined by the claims.

HDMR Re-Ordering as Applied to Site-Directed Protein Mutagenesis

Most protein mutation studies to date have been highly selective, involving low-order multiple mutations guided by intuition about protein chemistry and amino acid physical/chemical properties. Rarely have such studies systemically analyzed a large set of both single and multiple mutants needed to evaluate the effectiveness of HDMR re-ordering. Some systematic data are available for the gene V protein of bacteriophage f1, which is a homodimeric single-stranded DNA-binding protein (Sandberg et al., Proc. Natl. Acad. Sci. USA, 90, 8367-8371 (1993); Sandberg et al., Biochem., 34, 11970-11978 (1995)). Two contacting positions within the hydrophobic core of the protein were partially randomized, Val35 and Ile47, with eight replacements being made at each position, including many of the corresponding double mutants. The activity of the mutants was assessed semi-quantitatively by the degree of temperature-sensitivity of their phage growth phenotype, and all of the single mutants plus 18 of the double mutants were purified and characterized with respect to both folding stability and DNA binding affinity.

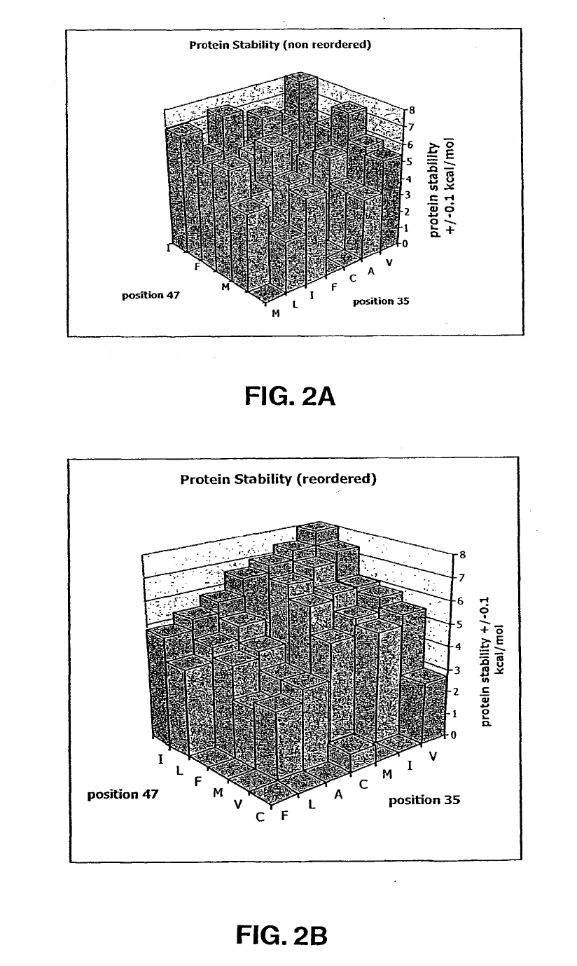

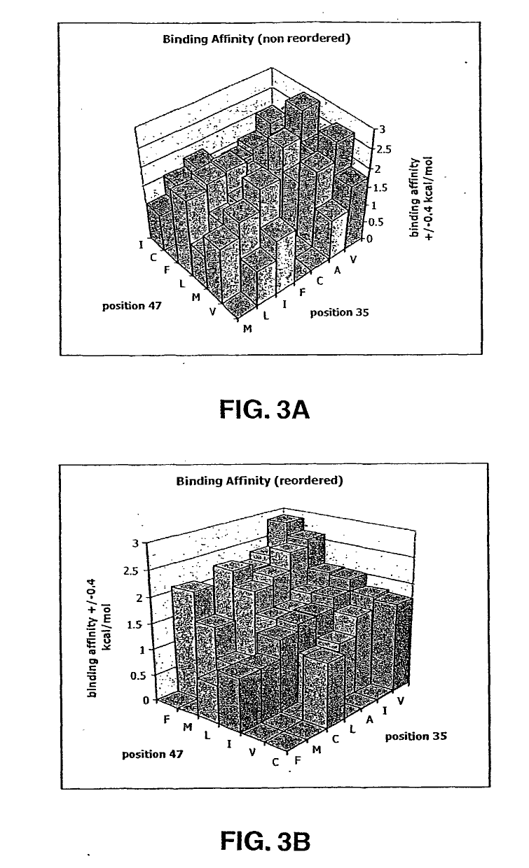

A relatively sparse but still informative matrix can be plotted using the stability and binding data of Sandberg et al. (1995), as shown in FIGS. 2A, 2B, 3A and 3B, respectively. Re-ordering the variables based on the laboratory data using the wildtype protein as the cut center demonstrates that regular patterns can be identified. FIGS. 2A and 3A use the original ordering of the residue replacements given by Sandberg et al. (i.e., presented in tabular form). Despite the fact that the effects of single-site replacements were relatively modest and the majority of pairwise substitutions gave additive effects, no pattern is evident in the plots of FIGS. 2A and 3A. In contrast, FIGS. 2B and 3B show that re-ordering response data to generate monotonic behavior along each axis leads to a relatively smooth response over the full space. In the general case, the full-space response surface need not be monotonic as it largely is here, and in the presence of significant non-additive effects, it will likely not be. However, the response surface need only be reasonable regular to enable the use of interpolation and HDMR analysis over the full space. An important consideration in such studies, especially for cases with mutations at many sites, is the accuracy of the data Although HDMR provides a systematic means to coarsely sample and interpolate over the full space of possible mutations, the accuracy of the observed data is critical. One conclusion from HDMR is that fewer mutations should provide an estimate for the protein functional properties throughout the space, provided that due attention is paid to the quality of the observations.

As is evident in the example of the gene V protein, each functional property can have a different monotonic re-ordering of the variables, and thus will have its own unique pattern for the relative significance of the terms in the HDMR expansion. As well, the consequent regularity of the response surface after re-ordering the variables implies the existence of patterns in the properties of the variables (side chain types). Interpretation of data guided by this method could conceivably result in a more comprehensive understanding of the properties of the individual residues that are relevant for various molecular functions or properties.

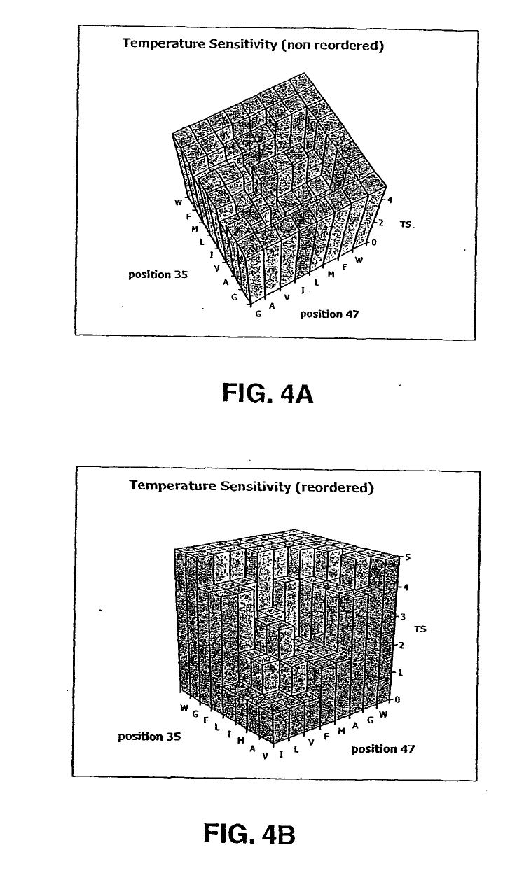

The method of variable reordering followed by HDMR analysis is not limited to protein mutation studies, but can be extended to other types of molecules and their related observable properties (e.g., pharmaceuticals and genomics). Furthermore, this method of re-ordering does not require a priori understanding of the relationship between the constituents and the observed property, but instead allows the observed property to define the relationship. As an example, FIGS. 4A and 4B depict the semi-quantitative in vivo temperature sensitivity data for the mutants of Sandberg et al. Just as for the binding and stability data in FIGS. 2A, 2B, 3A and 3B, re-ordering brings regularity to the response surface despite our lack of understanding of the molecular basis for the temperature-sensitive phenotype.

These results suggest the generality of the reordering approach, and suggest its applicability to a broad range of other types of multidimensional data analysis. As will be readily appreciated, numerous variations and combinations of the features set forth above can be utilized without departing from the present invention as set forth in the claim. Such variations are not intended as a departure from the spirit and scope of the invention, and all such variations are intended to be included within the scope of the following claims.

1. A method for the selective variation of a multi-variable molecular synthesis to optimize a functional property of the reaction product, comprising:
constructing a first order library of functional property output data obtained by reacting all first order library input variable combinations for said multi-variable synthesis and measuring said functional property for every reaction product, wherein said first order library input variable combinations comprise all values selected for each variable taken one at a time while the other variable values are held constant or randomized;
ordering the values for each input variable according to their effect upon functional property optimization based upon said first order library functional property output data;
constructing a second order library of functional property output data obtained by reacting a set of second order library input variable combinations coarsely sampled from said ordered input variables and measuring said functional property for every reaction product, wherein said second order library input variable combinations are assembled two variables at a time from said coarse sampling of ordered input variables while the other variable values, if any, are held constant or randomized; and
interpolating among said functional property output data for optimization of said functional property.

2. The method of claim 1, further comprising the step of selecting the reaction product with the optimal functional property from the results of said interpolating step.

3. The method of claim 1, wherein, when constructing said first order library, at least one input variable is randomized among the combinations.

4. The method of claim 3, wherein, when constructing said first order library, said input variables are all fully randomized among the combinations.

5. The method of claim 1, wherein said multi-variable synthesis reaction comprises the synthesis of molecules to be investigated for pharmaceutical activity, and said input variables include one or more chemical scaffolds and sets of moieties to be varied at sites of chemical functionalization on said scaffolds.

6. The method of claim 1, wherein said multi-variable synthesis reaction comprises the synthesis of proteins for the investigation of site-directed mutagenesis, and said input variables include sets of amino acid residues to be incorporated at each mutagenesis site.

7. The method of claim 1, wherein at least one input variable is a reaction condition.

8. The method of claim 1, wherein after said step of ordering the values for each input variable according to their effect upon functional property optimization, said method further includes the steps of selecting additional input variable combinations by interpolation of data relative to functional property optimization for at least one input variable, adding said additional input variable combinations to said first order library, and repeating said steps of constructing said first order library of functional property output data and ordering the values for each input variable before constructing said second order library.

9. The method of claim 8, wherein said additional input variable combinations comprise second order combinations.

10. The method of claim 8, wherein said additional input variable combinations comprise new variable selections.

11. The method of claim 1, wherein the coarse sampling is an ordered coarse sampling.

12. The method of claim 11 wherein said ordered coarse sampling is non-uniform

13. The method of claim 1, wherein said coarse sampling is a random coarse sampling.

14. The method of claim 13, wherein said random coarse sampling is non-uniform.

15. The method of claim 1, further comprising the steps of identifying the input variable combination producing the reaction product having the optimum functional property and reacting said optimum input variable combination to synthesize said reaction product.

16. The method of claim 1, wherein after said interpolating step, said method further includes the steps of (1) expanding said second order library of functional property output data by (a) coarsely sampling more second order input variable combinations from said ordered input variables, (b) reacting said additional second order input variable combinations, and (c) measuring said functional property for every reaction product; and (2) repeating said interpolating step.

17. The method of claim 16, wherein said steps of expanding said second order library of functional property output data and repeating said interpolating step are performed iteratively.

18. The method of claim 17, further including the step of repeating said step of ordering the values for each input variable according to their effect upon functional property optimization based upon both the first order library and second order library functional property output data.

19. The method of claim 18, wherein before repeating said step of ordering the values for each input variable, said method further includes the step of repeating said step of constructing a first order library in order to obtain additional first order library functional property output data.

20. The method of claim 1, wherein after said interpolating step, said method further includes the steps of (1) making correlated second order input variable combinations based on said second order library functional property output data; and (2) repeating said steps of constructing said second order library of functional property output data and interpolating among said functional property output data.

21. The method of claim 1, wherein said functional property output data comprises output data for more than one functional property with the variable values being independently ordered with respect to each functional property.

22. The method of claim 1, wherein after said interpolating step, said method further includes the step of expanding the number of values selected for at least one variable and repeating said method from the construction of said first order library.

23. The method of claim 1, wherein after said interpolating step, said method further includes the steps of constructing a third order library of functional property output data obtained by reacting a set of third order library input variable combinations coarsely sampled from said ordered input variables and measuring said functional property for every reaction product, wherein said third order library input variable combinations are assembled three variables at a time from said coarse sampling of ordered input variables while the other variable values, if any, are held constant or randomized; and interpolating among said functional property output data for optimization of said functional property.
